(19) United States
(12) Patent Application Publication
(10) Pub. No.: US 2026/0315479 A1
(43) Pub. Date: Oct. 15, 2026

(54) PROGESTOGEN-FREE ORAL DOSAGE UNIT FOR USE IN FEMALE CONTRACEPTION AND TREATMENT OF ENDOMETRIOSIS

(71) Applicant: Pandora Endocrine Innovation B.V., Bergen (NL)

(72) Inventor: Herman Jan Tijmen Coelingh Bennink, Bergen (NL)

(73) Assignee: Pandora Endocrine Innovation B.V., Bergen (NL)

(21) Appl. No.: 19/485,289

(22) PCT Filed: May 21, 2024
(86) PCT No.: PCT/EP2024/063987

§ 371 Date: Nov. 18, 2025

(30) Foreign Application Priority Data
May 26, 2023 (EP) 23175829.3

(51) Int. Cl. A61K 31/519; A61K 31/513; A61K 31/565; A61K 31/5685; A61K 31/57; A61P 15/18

(52) U.S. Cl. CPC A61K 31/519; A61K 31/513; A61K 31/565; A61K 31/5685; A61K 31/57; A61P 15/18

(57) ABSTRACT

The invention relates to an oral dosage unit for use in a method of contraception in a mammalian female or for use in a method of treating endometriosis in a mammalian female, said oral dosage unit containing GnRH antagonist, estrogen and no progestogen; and wherein said method comprises once daily oral administration to the mammalian female of the oral dosage unit during a period of at least 28 days.

### TECHNICAL FIELD OF THE INVENTION

The present invention relates to a progestogen-free oral dosage unit for use in female contraception and treatment of endometriosis. The oral dosage unit of the present invention comprises a combination of a gonadotropin-releasing hormone (GnRH) antagonist and an estrogen.

### BACKGROUND OF THE INVENTION

Currently, there are two types of oral contraceptive pills: combined estrogen-progestogen and progestogen-only. The most commonly prescribed pill is the combined hormonal pill with estrogen and progestogen, usually referred to as combined oral contraceptive (COC). Simply put, progestogen is the hormone that inhibits ovulation and thereby prevents pregnancy, and the estrogen component controls menstrual bleeding and substitutes the loss of the endogenous ovarian estrogen estradiol (E2).

Depending on withdrawal bleeding desired by the patient and clinically recommended, COC can be prescribed as a cyclic (monthly bleeding), extended cyclic (every three months bleeding), or continuous dosing (no bleeding).
Cyclic formulations: The cyclic formulations have active hormone pills for 21-24 days, followed by 7-4 days of hormone-free pills.
Extended cycle formulations: extended cycle formulations have active hormone pills every day for three months, followed by a placebo week.
Continuous use formulations: can be manipulated by using the only active pills from monthly formulations for one year period, which will functionally stop all menstrual bleeding.

It is common practice to treat endometriosis with hormone therapy. Many doctors prescribe a combination of a progestogen with estrogen add-back.

Progesterone from normal menstrual cycles is highly correlated with the cause of breast cancer as shown in a Perspective Review Paper (Herjan C. T. Coelingh Bennink et al, Progesterone from ovulatory menstrual cycles is an important cause of breast cancer, Breast Cancer Research (2023), 25:60). The authors of this article reviewed the literature regarding the relationship between ovulatory menstrual cycles (MCs) and breast cancer (BC) risk. The authors postulate that progesterone is an important factor in a woman's lifetime risk of developing BC, and that breast tumours arising during hormonal contraception or after menopause, with or without menopausal hormone therapy, are the consequence of the outgrowth of pre-existing neoplastic lesions, eventually stimulated by estrogens and some progestins. According to the authors, their analysis of clinical and molecular data suggests that progesterone and not estradiol or testosterone is an important cause of BC by stimulating proliferation of normal breast epithelium during the luteal phase of the MC through the paracrine factors WNT4 and RANKL. Progesterone also appears to upregulate the expression of the DNA mutator enzyme APOBEC3B during this phase of the MC. These effects of progesterone likely increase the accumulation of mutations in long-lived mammary stem and progenitor cells. Estrogens, testosterone (as precursor of estradiol) and most environmental factors that are related to the risk of BC may stimulate the growth of already existing ER+/PR+BC, but have only mild proliferative effects on normal breast epithelium and, in light of our hypothesis, are therefore unlikely to cause BC. The authors propose to develop medical strategies in women that avoid exposure to natural progesterone and progestins as much as possible.

Gonadotropin-releasing hormone (GnRH) antagonists are a class of medications that antagonise the GnRH receptor by competing with natural GnRH for binding to GnRH receptors, thus decreasing or blocking GnRH action in the body. GnRH antagonists are used in the treatment of prostate cancer, endometriosis, uterine fibroids, female infertility in assisted reproduction, and for other indications. Currently approved GnRH antagonists include the peptide molecules abarelix, cetrorelix, degarelix, and ganirelix and the small-molecule compounds elagolix and relugolix. GnRH antagonists are administered by subcutaneous injection (cetrorelix, degarelix, ganirelix), by intramuscular injection (abarelix), or by oral administration (elagolix, relugolix). Another non-peptide and orally-active GnRH antagonist that is in development is linzagolix.

WO 87/05514 relates to a delivery system for preventing pregnancy in a female mammal, comprising:
a first delivery system for administration to said mammal during the follicular phase of the menstrual cycle, of an effective amount of an LHRH composition and an effective amount of an estrogenic steroid; and,
a second delivery system for administration to said mammal during the luteal phase of the menstrual cycle, an effective amount of an LHRH composition, an effective.amount of an estrogenic steroid, and an effective amount of progestational steroid.

Fraser (GnRH analogues for contraception, British Medical Bulletin (1993) Vol 49, No 1, pp 62-72) discusses the use of GnRH analogues in contraception. The abstract of the article reads as follows: The production of chemical analogues of GnRH permits direct suppression of the pituitary-gonadal axis at the level of the gonadotroph. Continuous administration of GnRH agonists desensitises the gonadotroph and ovulation is uniformly prevented, forming the practical basis for use in contraception. However, long-term treatment is constrained by variable effects on oestrogen secretion, which cause irregular bleeding patterns on the one hand and risks of hypo-estrogenism on the other. Their use as a post-partum contraceptive has attractions because any analogue in milk should be without biological activity in the infant. GnRH antagonists have the advantage of immediate inhibitory action. They have potential application in circumstances in which agonists have been employed and, in addition, can interrupt any stage of the menstrual cycle. Clinical trials to utilise their potential antifertility action have not been performed. Use of GnRH analogues for contraception in women may require combination with low dose oestrogen and progestin and it has been proposed that such development may yield important benefits in health. When combined with testosterone, GnRH antagonists may form the basis for a male contraceptive.

Ryeqo® is a medicine used in women of reproductive age to treat moderate to severe symptoms of uterine fibroids and symptoms of endometriosis. Ryeqo contains 40 mg relugolix, 1 mg estradiol (hemihydrate) and 0.5 mg norethisteronacetate. After at least one month of Ryeqo use, it inhibits ovulation in women taking the recommended dose and provides adequate contraception.

### SUMMARY OF THE INVENTION

The inventor has devised an oral dosage unit that is progestogen-free and that can suitably be used as a continuous use formulation to prevent ovulation and/or to maintain amenorrhea during a period of 28 days or longer. Thus, the oral dosage unit can suitably be used as a female oral contraceptive and in the treatment of endometriosis.

Accordingly, one aspect of the invention relates to an oral dosage unit for use in a method of contraception in a mammalian female or for use in a method of treating endometriosis in a mammalian female, said oral dosage units containing GnRH antagonist, estrogen and no progestogen; and wherein said method comprises once daily oral administration to the mammalian female of the oral dosage unit during a period of at least 28 days.

In comparison to known female oral contraceptives, the female oral contraceptive of the present invention offers a combination of advantages. First of all, since the oral dosage units do not contain progestogen, an increased breast cancer risk associated with progestogens is avoided. Secondly, since the contraceptive method of the present invention induces prolonged amenorrhea, the frequency of menses and the associated complaints of the menstrual cycle are negated dramatically.

Compared to hormone therapy of endometriosis that employs a combination of progestogen and estrogen to maintain amenorrhea, the present method of treating endometriosis offers the advantage that an increased breast cancer risk associated with progestogens is avoided.

Another aspect of the invention relates to a dosage unit comprising:
GnRH antagonist in an amount equivalent to 100-400 mg elagolix;
estrogen in an amount equivalent to 2.5-25 μg ethinyl estradiol; and
no progestogen.

### DETAILED DESCRIPTION OF THE INVENTION

As mentioned above, a first aspect of the invention relates to an oral dosage unit for use in a method of contraception in a mammalian female or for use in a method of treating endometriosis in a mammalian female, said oral dosage units containing GnRH antagonist, estrogen and no progestogen; and wherein said method comprises once daily oral administration to the mammalian female of the oral dosage unit during a period of at least 28 days.

The term “GnRH antagonist” as used herein refers to a class of medications that bind to the GnRH receptor without activating said receptor.

The term “estrogen” as used herein refers to a class of natural or synthetic steroid hormones that bind to and activate the estrogen receptors.

The term “estradiol” as used herein refers to estra-1,3,5 (10)-triene-3, 17β-diol.

The term “estriol” as used herein refers to estra-1,3,5 (10)-triene-3, 16a, 17β-triol.

The term “estetrol” as used herein refers to estra-1,3,5 (10)-triene-3, 15a, 16a, 17β-triol.

The term “ethinyl estradiol” as used herein refers to 17α-Ethynylestra-1,3,5 (10)-triene-3, 17β-diol.

The term “progestogen” as used herein refers to a class of natural or synthetic steroid hormones that bind to and activate the progesterone receptor.

The term “prodrug” as used herein refers to a substance that, after oral administration, is metabolised into a pharmaceutically active drug. Thus, for instance, a prodrug of estradiol is a substance that is metabolised into estradiol after oral administration. Estradiol valerate is an example of a prodrug of estradiol.

The method of the present invention preferably comprises administration of the oral dosage unit to a human female, more preferably a human female of childbearing capability.

In accordance with a particularly preferred embodiment, the once daily oral administration of the oral dosage unit in accordance with the present method prevents ovulation.

In accordance with another particularly preferred embodiment, the once daily oral administration of the oral dosage unit in accordance with the present method maintains amenorrhea.

According to a particularly preferred embodiment, the method comprises once daily oral administration of the oral dosage unit during a period of at least 56 days, more preferably of at least 84 days.

The GnRH antagonist that is employed in accordance with the present invention is preferably selected from relugolix, elagolix, linzagolix and combinations thereof. More preferably, the GnRH antagonist is selected from relugolix and linzagolix and combinations thereof. Most preferably, the GnRH antagonist is relugolix.

Preferably, in the present method GnRH antagonist is orally administered in a daily dosage that is equivalent to a daily oral dosage of 20 to 200 mg relugolix, more preferably of 30 to 120 mg relugolix. In the following table the equivalent dosages are listed for a number of GnRH antagonists that may be employed in accordance with the present invention.

Equivalent to

| | 20-200 mg | 30-120 mg |
|---|---|---|
| | relugolix per day | relugolix per day |
| Elagolix | 67-667 mg per day | 100-400 mg per day |
| Linzagolix | 45-450 mg per day | 78-270 mg per day |

In an embodiment of the invention the GnRH antagonist is relugolix. The oral dosage unit preferably contains 20 to 200 mg relugolix, more preferably 30 to 120 mg relugolix, most preferably 35-80 mg relugolix.

In another embodiment, the GnRH antagonist is elagolix. The oral dosage unit preferably contains 60 to 600 mg elagolix, more preferably 100 to 400 mg elagolix and most preferably 150-300 mg elagolix.

In yet another embodiment, the GnRH antagonist employed is linzagolix. Preferably, the oral dosage unit contains 50 to 400 mg linzagolix, more preferably 75 to 300 mg linzagolix, most preferably 80-300 mg linzagolix.

The estrogen that is employed in accordance with the present invention is preferably selected from ethinyl estradiol, estradiol, a prodrug of estradiol, estriol, a prodrug of estriol, estetrol, a prodrug of estetrol and combinations thereof. More preferably, the estrogen is selected from ethinyl estradiol, estradiol, estradiol valerate, estetrol and combinations thereof. Even more preferably, the estrogen is selected from ethinyl estradiol, estradiol, estetrol and combinations thereof. In one particularly preferred embodiment, the estrogen employed in the present method is ethinyl estradiol. In another particularly preferred embodiment, the estrogen employed is estetrol.

According to a particularly preferred embodiment of the present method estrogen is administered in a very low dose that is sufficient to prevent symptoms of hypoestrogenism but low enough not to stimulate the endometrium and cause irregular bleeding.

Accordingly, the estrogen is preferably orally administered in a daily dosage that is equivalent to a daily oral dosage of 3-30 μg ethinyl estradiol, more preferably of 5-15 μg ethinyl estradiol. In the following table the equivalent dosages are listed for a number of estrogens that may be employed in accordance with the present invention.

Equivalent to

| | 2.5-25 μg ethinyl | 5-15 μg ethinyl |
|---|---|---|
| | estradiol per day | estradiol per day |
| Estradiol | 0.25-2.5 mg per day | 0.5-1.5 mg per day |
| Estradiol valerate | 0.3-3.0 mg per day | 0.6-2.0 mg per day |
| Estriol | 10-100 mg per day | 20-50 mg per day |
| Estetrol | 7.5-75 mg per day | 15-45 mg per day |

In an advantageous embodiment of the present method, the oral dosage unit contains ethinyl estradiol, more preferably 2.5-25 μg ethinyl estradiol, and most preferably 5-15 μg ethinyl estradiol

In another advantageous embodiment, the oral dosage unit contains estradiol and/or a prodrug of estradiol. More preferably, the oral dosage unit contains 0.25-2.5 mg estradiol and/or it contains a prodrug of estradiol in an amount equivalent to 0.25-2.5 mg estradiol. Most preferably, the oral dosage unit contains 0.5-1.5 mg of estradiol and/or it contains a prodrug of estradiol in an amount equivalent to 0.5-1.5 mg estradiol.

In yet another advantageous embodiment, the oral dosage unit contains estriol. More preferably, the oral dosage unit contains 10-100 mg of estriol. Most preferably, the oral dosage unit contains 20-50 mg of estriol.

In a further advantageous embodiment, the oral dosage unit contains estetrol. More preferably, the oral dosage unit contains 7.5-75 mg of estetrol. Even more preferably, the oral dosage unit contains 15-45 mg of estetrol. Most preferably, the oral dosage unit contains 20-40 mg of estetrol.

Due to the action of the GnRH antagonist, the present method suppresses secretion of luteinizing hormone/follicle stimulating hormone secretion and thereby also suppresses endogenous production of estradiol and testosterone. In order to prevent negative effects resulting from lowered testosterone levels, the oral dosage unit used in the present method preferably contains 15-150 mg, more preferably 25-75 mg dehydroepiandrosterone.

In order to minimise the risk or irregular bleeding, the method of the present invention preferably comprises intra-uterine co-administration of progestogen. Preferably, the progestogen employed is levonorgestrel. According to a particularly preferred embodiment, levonorgestrel is co-administered using an intra-uterine device releasing 3-25 μg levonorgestrel per day.

Another aspect of the invention relates to an oral dosage unit comprising:
GnRH antagonist in an amount equivalent to 100-400 mg elagolix;
estrogen in an amount equivalent to 2.5-25 μg ethinyl estradiol; and
no progestogen.

Preferably, the GnRH antagonist in the oral dosage unit is selected from relugolix, elagolix, linzagolix and combinations thereof. More preferably, the GnRH antagonist is selected from relugolix and linzagolix and combinations thereof. Most preferably, the GnRH antagonist is relugolix.

The amount of GnRH antagonist in the oral dosage unit is preferably equivalent to 20 to 200 mg relugolix, more preferably of 30 to 120 mg relugolix. The equivalent dosages for a number of GnRH antagonists that may be employed in the oral dosage unit have been listed above.

In an embodiment of the oral dosage unit of the present invention the GnRH antagonist is relugolix. The oral dosage unit preferably contains 20 to 200 mg relugolix, more preferably 30 to 120 mg relugolix, most preferably 35-80 mg relugolix.

In another embodiment, the GnRH antagonist is elagolix. The oral dosage unit preferably contains 60 to 600 mg elagolix, more preferably 100 to 400 mg elagolix and most preferably 150-300 mg elagolix.

In yet another embodiment, the GnRH antagonist employed is linzagolix. Preferably, the oral dosage unit contains 45 to 450 mg linzagolix, more preferably 50 to 400 mg linzagolix, most preferably 80-300 mg linzagolix.

The estrogen in the oral dosage unit is preferably selected from ethinyl estradiol, estradiol, a prodrug of estradiol, estriol, a prodrug of estriol, estetrol, a prodrug of estetrol and combinations thereof. More preferably, the estrogen is selected from ethinyl estradiol, estradiol, estradiol valerate, estetrol and combinations thereof. Even more preferably, the estrogen is selected from ethinyl estradiol, estradiol, estetrol and combinations thereof. In accordance with one particularly preferred embodiment, the estrogen employed in the present method is ethinyl estradiol. In another particularly preferred embodiment, the estrogen employed is estetrol.

The amount of estrogen in the oral dosage unit is preferably equivalent to 2.5-25 μg ethinyl estradiol, more preferably to 5-15 μg ethinyl estradiol. The equivalent dosages for a number of estrogens that may be employed in the oral dosage unit have been listed above.

In an advantageous embodiment, the oral dosage unit contains ethinyl estradiol, more preferably 2.5-25 μg ethinyl estradiol, and most preferably 5-15 μg ethinyl estradiol

In another advantageous embodiment, the oral dosage unit contains estradiol and/or a prodrug of estradiol. More preferably, the oral dosage unit contains 0.25-2.5 mg estradiol and/or it contains a prodrug of estradiol in an amount equivalent to 0.25-2.5 mg estradiol. Most preferably, the oral dosage unit contains 0.5-1.5 mg of estradiol and/or it contains a prodrug of estradiol in an amount equivalent to 0.5-1.5 mg estradiol.

In yet another advantageous embodiment, the oral dosage unit contains estriol. More preferably, the oral dosage unit contains 10-100 mg of estriol. Most preferably, the oral dosage unit contains 20-50 mg of estriol.

In a further advantageous embodiment, the oral dosage unit contains estetrol. More preferably, the oral dosage unit contains 7.5-75 mg of estetrol. Even more preferably, the oral dosage unit contains 15-45 mg of estetrol. Most preferably, the oral dosage unit contains 20-40 mg of estetrol.

The oral dosage unit of the present invention preferably contains 15-150 mg, more preferably 25-75 mg dehydroepiandrosterone.

According to a particularly preferred embodiment, the dosage unit of the present invention comprises:
30-120 mg relugolix;
15-45 mg estetrol;
25-75 mg dehydroepiandrosterone; and
no progestogen.

Another aspect of the present invention relates to a package comprising at least 28 oral dosage units according to the present invention. More preferably, the package comprises at least 56 oral dosage units, most preferably 84-168 dosage units.

Preferably, the oral dosage units are arranged within the package in a fixed sequence, the total number of dosage units in the package being a multiple of 7.

Preferably, the oral dosage units are accommodated separately within the package.

According to a particularly preferred embodiment, date indications are provided adjacent to each oral dosage unit.

According to a particularly preferred embodiment, the oral dosage units are arranged in a blister pack.

The invention is further illustrated by the following non-limiting examples.

### EXAMPLES

### Example 1

In a multicentre, randomised, clinical trial, the efficacy and safety of three different oral contraceptives (OC) regimens in the prevention of pregnancy in sexually active women, ages 18 through 35 years, is evaluated. Subjects are randomized in a 1:1:1 fashion to one of the following regimens:
Relugolix (80 mg)/ethinyl estradiol (10 μg) administered once daily for 182 days as a combination oral tablet
Relugolix (80 mg)/ethinyl estradiol (5 μg) administered once daily for 182 days as a combination oral tablet
Linzagolix (150 mg)/ethinyl estradiol (10 μg) administered once daily for 182 days as a combination oral tablet

All subjects, regardless of randomization, initiate study OC therapy on the first Sunday following the beginning of their menstrual period (“Sunday starters”) and remain as Sunday starters throughout the study. All subjects complete and download information entered into an electronic diary. Assessments include study drug compliance, use of additional forms of contraception, bleeding patterns, weight, assessment of the incidence and severity of menstrual related symptoms and medication taken to relieve these symptoms. Information are self-recorded on the electronic diary via a series of pre-programmed questions.

Two hundred (200) subject in each treatment arm are targeted to complete the study. Subjects must meet the following criteria to be included in the study:
1. Sexually active adult females (age 18 through 35), of child bearing potential, in a heterosexual relationship, at risk for pregnancy, who are in good health and who have a history of OC use for an interval of at least three successive cycles with regular withdrawal bleeding (bleeding during the pill-free interval or during the first three days of the subsequent cycle) prior to enrollment (Continuous Users)
OR
have no prior history OC use (Fresh-Starts)
OR
have no history of OC use in the 6 months prior to enrollment (Prior Users)
2. Negative urine pregnancy test.
3. Signed informed consent.
4. Agree to use study oral contraceptive therapy as their primary birth control method (BCM).

Subjects are excluded from the study if any of the following criteria are met:
1. History of hypersensitivity to estrogen or progestin components of OCs.
2. History of alcohol or drug abuse.
3. Chronic use of any medication that may interfere with the efficacy of oral contraceptives.
4. History of being HIV or Hepatitis C positive.
5. History of persistent noncompliance with any chronic medication.
6. History of having received injectable hormone therapy within the 10 months prior to enrollment or having a progestin-releasing intrauterine device (IUD) in place within 3 months prior to enrollment or has had a contraceptive implant removed within one month prior to enrollment or has received any other form of hormonal contraception within 3 months prior to enrollment.
7. Routine concomitant use of additional forms of contraception (IUD, diaphragm, contraceptive sponge) with the exception of condoms.
8. Subjects who have had recent surgical or medical abortion, miscarriage, or vaginal or cesarean delivery must have had at least two normal menstrual cycles prior to enrollment.
9. History of abnormal bleeding (breakthrough or withdrawal bleeding that lasts ≥10 consecutive days or excessive spotting that lasts ≥10 consecutive days) while on conventional oral contraceptives.
10. History of thromboembolic disorder, vascular disease, cerebral vascular or coronary artery disease.
11. Uncontrolled or untreated hypertension (systolic BP≥140 mmHg and diastolic BP=90 mmHg on more than two occasions).
12. Known or suspected carcinoma of the breast, endometrial carcinoma or known or suspected estrogen dependent neoplasia.
13. Undiagnosed abnormal genital bleeding.
14. History of hepatic adenomas or carcinomas.
15. History of cholestatic jaundice of pregnancy or jaundice with prior OC use.
16. Known or suspected pregnancy or currently breastfeeding.
17. Hyperlipidemia requiring active treatment with antihyperlipidemic agents.
18. History of diabetes mellitus, glucose intolerance or gestational diabetes.
19. History of abnormal laboratory value at screening
20. Any clinically significant abnormal finding or condition on history, screening, physical exam, pelvic exam or any laboratory finding which contraindicates the use of oral contraceptives.
21. Has participated in any clinical investigation within the 30 days prior to enrolment.
22. Has donated or had a loss of more than 500 cc of blood within the 30 days prior to enrollment.

All subjects are instructed to take one tablet per day at approximately the same time each day.

A complete physical and gynaecologic exam, including PAP smear, is performed at screening and at the completion of therapy or upon early withdrawal from the study. Any subject with an abnormal Pap smear is disqualified for enrolment, unless investigator decides the results are not clinically significant and will not interfere with conduct of the study. The Investigator's decision is documented. Subjects who have had a Pap smear reported as within normal limits within the three months prior to enrollment in the study are not be required to have the test repeated. A copy of the results will be available in the subject's medical record. Any subject with a report of insufficient cells must have the test repeated and documented by the investigator as within normal limits prior to enrollment.

Clinical laboratory tests are performed at screening and at the completion of therapy or upon early withdrawal. All clinical laboratory tests are done at one central laboratory. Laboratory tests include CBC, serum chemistry, lipid profile, urinalysis, and urine pregnancy test.

All subjects are followed for the occurrence of pregnancy for three months following completion of the study. This follow-up may be in the form of a telephone call. All pregnancies that occur during the course of the study or in the three months following completion of the study are dated using ultrasound to establish the gestational age of the foetus.

Subjects are asked to complete electronic diaries. The diary is programmed to ask specific questions related to the study compliance, bleeding pattern and occurrence of symptoms that are commonly associated with the hormone fluctuation during the menstrual cycle. The questions address compliance, bleeding pattern and hormone-related symptoms.

No significant toxicity is expected from the study medication. However, if the subject develops any symptoms or any abnormal laboratory parameter attributed to the drug, which are considered by the subject and/or physician to be of unacceptable severity, then the study medication is discontinued.

Subjects are queried regarding concomitant medication use at monthly phone calls. All concomitant medication use (both prescription and over-the-counter (OTC), including herbal medications and nutritional supplements) must be reported during the study, and recorded on the subject's Case Report Form (CRF).

Subjects who require the initiation of chronic therapy with drugs that are known to interact with OCs are withdrawn from the study. Subjects who require intermittent therapy with drugs known to interact with OCs (e.g. antibiotic therapy) will remain in the study and will receive counselling regarding the need for additional contraceptive protection during the entire cycle. Subjects are provided with the list of medications that are known to interact with OC and are instructed to notify study coordinator as soon as medication is prescribed to receive proper counselling. Those cycles in which drugs known to interact with OC therapy are taken are not be used in the calculation of the pregnancy rate.

The results of the study show that the tested oral contraceptives regimens effectively prevent pregnancy in sexually active women without significant adverse side effects.

### Example 2

An oral dosage that is particularly suited for use in a contraceptive method according to the present invention is progestogen-free and contains:
50 mg relugolix;
25 mg estetrol; and
50 mg dehydroepiandrosterone

1.-28. (canceled)

29. A method of contraception in a mammalian female using oral dosage units, said oral dosage units containing:
GnRH antagonist in an amount equivalent to 20 to 200 mg relugolix;
estrogen in an amount equivalent to 2.5-25 μg ethinyl estradiol; and
no progestogen;
the method comprising once daily oral administration to the mammalian female of the oral dosage unit during a period of at least 28 days.

30. The method of contraception according to claim 29, wherein the oral dosage units contain GnRH antagonist selected from:
20 to 200 mg relugolix;
67 to 667 mg elagolix;
50 to 400 mg linzagolix;

31. The method of contraception according to claim 30, wherein the oral dosage units contain 20 to 200 mg relugolix.

32. The method of contraception according to claim 30, wherein the oral dosage units contain 67 to 667 mg elagolix.

33. The method of contraception according to claim 30, wherein the oral dosage units contain 50 to 400 mg linzagolix;

34. The method of contraception according to claim 29, wherein the oral dosage units contains estrogen selected from ethinyl estradiol, estradiol, estradiol valerate, estetrol and combinations thereof.

35. The method according to claim 34, wherein the oral dosage unit contains estrogen selected from:
2.5-25 μg ethinyl estradiol;
0.25-2.5 mg estradiol;
0.3-3.0 mg estradiol valerate;
10-100 mg estriol; and
7.5-75 mg estetrol.

36. The method according to claim 35, wherein the oral dosage unit contains 7.5-75 mg estetrol.

37. The method of contraception according to claim 29, wherein the once daily oral administration of the oral dosage unit induces amenorrhea.

38. The method of contraception according to claim 29, wherein the oral dosage unit contains 15-150 mg dehydroepiandrosterone.

39. The method of contraception according to claim 29, wherein the oral dosage unit comprises:
30-120 mg relugolix;
15-45 mg estetrol;
25-75 mg dehydroepiandrosterone; and
no progestogen.

40. The method of contraception according to claim 29, wherein the method comprises intra-uterine co-administration of progestogen.
